(19) United States
(12) Patent Application Publication
(10) Pub. No.: US 2024/0052359 A1
(43) Pub. Date: Feb. 15, 2024

(54) MULTIPLEX TARGETED RECOMBINATIONS FOR TRAIT INTROGRESSION APPLICATIONS

(71) Applicant: LIMAGRAIN EUROPE, Saint-Beauzire (FR)

(72) Inventors: Jean-Pierre MARTINANT, Vertaizon (FR); Pascual PEREZ, Chanonat (FR); Jean-Philippe PICHON, Clermont-Ferrand (FR)

(21) Appl. No.: 18/264,794

(22) PCT Filed: Feb. 10, 2022
(86) PCT No.: PCT/EP2022/053232

§ 371 Date: Aug. 9, 2023

(30) Foreign Application Priority Data
Feb. 10, 2021 (EP) 21305180.8

(51) Int. Cl. C12N 15/82; C12N 9/22

(52) U.S. Cl. CPC C12N 15/8241; C12N 15/8216; C12N 9/22; C12N 2310/20

(57) ABSTRACT

The invention relates to a plant containing genes coding for a dead Cas protein, a Spo11 protein, a guide RNAs around traits of interest and its use to improve introgression of the traits.

Trait Introgression (TI) process has been used for decades to transfer simple traits from a donor plant to an elite line newly obtained from a breeding project.

Such Trait Introgression corresponds to the introduction of gene(s), GM event(s) or locus (loci) responsible for the trait from a donor plant to a recipient plant, possibly an elite line. These genes, GM events or loci are herein called “genetic determinant associated with the trait”, since their presence in the genome of a plant leads to appearance of the phenotype (trait).

It is reminded that a trait is a morphological, physiological or phenological characteristic of an organism measured at the individual level that affects its individual performance.

The trait introgression process is widely used in vegetable and field seeds species to introgress (introduce) native alleles like disease resistance, quality traits (for instance oil or starch) and herbicide resistances in a new genotype. This step allows the addition of some properties expected by growers or consumers in a commercial cultivar. The commercial cultivar can be defined a line (or a hybrid plant) that has been optimized with regards to the conditions under which it is to be cultured.

Commercial cultivar or plant lines or varieties are obtained by breeding (also called selection) where specific phenotypic traits are obtained by choosing which which plant (males and females) are to be crossed and, selecting the offspring (progeny) that possess the most interesting genes, markers and/or phenotypes. After various rounds of crossing, self-pollination is generally performed to obtain homozygous plants.

Introgression consists in introducing a specific determinant (a gene, a locus (characterized by markers, or a transgene) in a line that already possesses a genome of interest. such line can be designated as the recurrent line. The process includes multiple steps of back-crossing (BC) (i.e. crossing plants from the progeny that possess the determinant, as well as the highest genome-ratio from the recurrent line with the recurrent line). Once plants of the progeny possess a high enough genome-ratio from the recurrent line and the desired trait, these plants are subjected to self-pollination (S). Generally, one would make about three to five back-crosses and two to three pollinations.

Trait Introgression is also a compulsory step for the production of GM cultivars since in such case it is generally not permitted to breed in the presence of GM events. Then, GM traits, like for example herbicide resistance or pest resistance, must be added at the end of the breeding process after the selection of the best lines for all the other characteristics including behavior in targeted environmental conditions. Clean introgression of the traits loci from a donor line is usually desired in the recipient line by minimizing the insertion of unwanted donor segments associated with linkage drag.

Introgression accuracy, in term of chromosomal physical length, as well as the time and the efforts required to achieve it is dependent on the location and frequency of recombination events naturally occurring during the plant gamete formation.

Indeed, increase of the genome ratio during the various steps of back-crossing is linked to crossing-over events (recombination events) that occur during meiosis and that allow to introduce, in the chromosome bearing the determinant, genome parts from the recurrent line. Such recombination events rate is thus directly linked to the rate of “crossing-over”. Crossing-over happens during meiosis when the paired chromosome homologs are lined up (prophase I of meiosis before tetrads are aligned along the equator in metaphase I). During this stage, recombination can occur between both homologous chromatids, (chromosomal crossover) at the point of contact between these two (non-sister) chromatids (chiasma). The result of this crossover is the exchange of the distal (from the chromatid centromere) ends of the two homologous chromosomes and the introduction of the distal end of one chromosome in place of the distal end of the homologous chromosome.

In theory, Trait Introgression is not complex, but it is a long and laborious process requiring robust logistics and a large number of plants.

The process has been greatly simplified by the use of molecular markers allowing for molecular detection of the presence of the genetic sequence of locus, responsible for the required trait, in a heterozygote background at a very early stage of plantlet development or even at the embryo level before any germination.

Nevertheless, in parallel to this efficiency improvement, the demand has been complexified with requirement to introgress multiple traits for the development of a commercial variety from a selected line. This is particularly true when multiple transgenes (GM events) are to be introduced in an elite line (for instance an elite line, the genetic background of which was developed for use in a specific environment).

In practice, TI process is usually done by dedicated teams in breeding organizations. These teams start from breeding selected lines (Recurrent) and cross them with a plant carrying the genetic determinants (genes, loci) associated with the trait(s) of interest (Donor). This is the first generation producing seeds which are called BC0. BC0 seeds germinate to give BC0 plants which are crossed back with the recurrent line to produce BC1 seeds (second generation). The same process is applied up to the production of BC3 seeds, i.e. the 4th generation. Afterward BC3 plants are usually selfed (crossed to themselves) two to three times to fix the genetic determinant responsible for the trait(s) and increase seeds.

Usually, several families phenotypically close to the initial elite recurrent line are eventually tested in normal growing conditions in order to identify the ones that are closer to the initial line derived from the breeding process.

As mentioned above, the use of molecular markers has improved a lot the efficiency of the TI process. Another improvement developed these last years is the growing of plants in control conditions to speed up the intergeneration time.

In order to limit the risk that the converted lines are not acceptable for the market, an important performance indicator for TI products is the genome similarity between the converted line and the recurrent line, i.e. the recurrent parent recovering percentage. To improve this indicator, during the process TI teams try to identify the plants with genetic recombination as close as possible on each side of each genetic determinant associated with the trait.

To elaborate a simple case, the introgression of one trait is generally done by the molecular identification of the 2%* of BC1 seeds or plantlets having the trait and a recombination at less than 2 cM (centiMorgan) on either side of the genetic determinant associated with the trait, followed by the identification of the 1%** of BC2 seeds or plantlets having the trait and a recombination at less than 2 cM on the other side of the trait. For this simple case to be safe 150 seeds could be considered at BC1 stage and 300 seeds at BC2 stage.

*BC1 seeds: 50% of them have the trait, 2% of which are recombined at their left border+2% of which are recombined at their right border

**BC2 seeds: 50% of them have the trait, 2% of them are recombined at the remaining border

If one considers simultaneous introgression of 2 traits, the number of seeds to be considered will be much higher if the same number of generations and the same specifications (i.e. recombination in the 2 cM region of the two traits borders) are to be examined.

To obtain one BC2 plant that has a recombinant event at both borders of trait 1 and of trait 2, it is reasonable to seek one recombination per trait at BC1 and to seek the recombinations at the remaining borders on the other side of the traits in BC2. To achieve this, one would need to screen in the order of 650,000 BC1 plantlets and in the order of 25,000 BC2 plantlets, based on a reasonable assumption of 100 BC2 kernels per harvested BC1 plant. This scenario is quite out of reach. If the recombination window is enlarged from 2 cM to 5 cM, figures fall significantly but nevertheless remain substantial: 20,000 BC1 and 4,000 BC2.

Usually, in such case of introgression of two traits, users accept to enlarge the window for recombination and take the risk to get a converted plant that is not compliant after the selfing of BC3 plants, or extend the conversion with one extra generation and derived products from BC4, or a combination of both, i.e. enlarge recombination window and add one generation.

When 3 traits are to be introgressed simultaneously, the breeders generally again accept more compromise on the length of remaining donor fragment(s) and the length of the process, even if, in such case, the extension of one generation for the trait introgression process could mean a one-year delay to reach the market. Such delay can have a huge negative impact on the net profit.

There are now new needs to introgress up to 5 traits simultaneously, especially for GM traits. Regular strategy (screening BC1 and BC2 seeds) can't be performed, as it would require increasing the number of BC1 and BC2 seeds to a number that isn't realistic.

In this case, TI teams introgress 3 traits on one hand (in one line) and the 2 remaining traits on the other hand (in another line) and combine the lines by crossing BC2 or BC3 plants (or BC4). This results in an increase of the volume of activity, the corresponding cost and time, in particular as there is a need of for more generations to obtain the final line.

### Definitions

A “trait” is a morphological, physiological or phenological characteristic of an organism that can be observed or measured at the individual level. Examples of traits include herbicide resistance, insect resistance, resistance to biotic or abiotic stress, or yield increase.

A “determinant” or “genetic determinant” is a gene, an allele, a GM event or a locus that is associated with the trait, as the presence of the determinant in the plant leads to occurrence of the trait. As an illustration, for insect resistance, the determinant can be a GM event (such as a bacterial gene that induces insect resistance).

A “dead Cas” protein is a Cas protein devoid of nucleolytic activity. Such protein is still able to bind to DNA with an appropriate gRNA, but lacks the ability to induce a strand break to the sequence to which it is bound.

A “guide RNA” or “gRNA” is a piece of RNAs that function as guides for RNA- or DNA-targeting enzymes, which they form complexes with. In the CRISPR/Cas system, the guide RNA will be used to direct the Cas protein to specific genome regions. It can thus be considered as “associated with the Cas protein”.

A “Spo11 protein” is a protein coded by the spo11 gene able to create double strand breaks to initiate meiotic recombination. Illustrations of Spo11 proteins are Uniprot Q9Y5K1 and Q5TCH6 (human) or Q9WTK8 (mouse). Other Spo11 proteins are RefSeq NP_036576, NP_937998 (human), or NP_001077428, NP_001077429, NP_001292363, NP_036176 (mouse). Yeast Spo11 is depicted in Uniprot P23179 (Saccharomyces cerevisiae). Spo11, and its function, have been conserved through evolution. One can also cite, as the Spo11 protein, a plant Spo11 protein, in particular selected from: Arabidopsis thaliana Spo11 proteins, such as described under the reference Uniprot Q9M4A2-1, Spo11 proteins from Oryza sativa (rice), such as described by Fayos I. et al. (Plant Biotechnol J. 2019 November; 17(11):2062-2077) and under the references UniProt Q2QM00, Q7Y021, Q5ZPV8, A2XFC1 and Uniprot Q6ZD95, Brassica campestris (mustard) Spo11 proteins, such as described under UniProt references A0A024AGF2 and A0A024AHI2, Zea mays (corn) Spo11 proteins, such as described under Uniprot references B6UAQ8 and B6TWI5, A0A1P8W169-1 and A0A1P8W163, the Spo11 proteins from Capsicum baccatum (pepper plant), such as described under references A0A2G2WFG5 and A0A2G2WFH4, the Spo11 protein from Carica papaya (papaya) such as described under Uniprot reference A0A024AG98. These sequences are described in WO2021234315. In the context of the invention, it is preferred to use plant Spo11 protein and gene, preferably from the same genus or species as the donor and recurring lines.

“Crossing-over” or “chromosome recombination” is a phenomenon that occurs during prophase at meiosis and relates to the genetic exchange between two homologous chromosomes. Homologous recombination is initiated with a break in both strands of one chromosome (a double-strand break), through Spo11, and recombination then occurs after an endonuclease enzyme cuts the chromosome that will “receive” the exchanged DNA. This meiotic homologous recombination leads to exchange of chromosomal regions of maternal and paternal origin, and generates genetic diversity in gametes.

Trait introgression is a mechanism based on crossing-over, in which it is desired to incorporate the genetic determinant associated to the trait from the chromosome of the donor plant to the chromosome of the receiving plant (recurrent line). The goal is to introduce as little as possible from the donor plant genome within the receiving plant genome.

The invention is based on the use of a dead Cas protein, in particular a dead Cas9 protein (or any DNA targeting protein) to target the Spo11 protein to sites that are in the 2 cM region on each side of the trait(s) to introgress, or preferably at about 50 kb, or even 10 kb from the trait, so as to foster and increase the occurrence of meiotic recombination events at these desired sites.

As indicated above, Spo11 creates double strand breaks to initiate meiotic recombination. The principle underlying the invention is thus to force double-strand breaks (DSB) during meiosis at sites that are close (between 1 cM and 5 cM, preferably between 1 and 2 cM, or between 10-1000 kb, preferably between 50-500 kb, or between 100-500 kb, more preferably between 50-100 kb) to the trait to introgress, in order to increase the frequency of recombination events, and hence increase the frequency of integration of the determinant of the trait within the genome of the recurrent line. This shall reduce the number of plants to screen at each back-cross step, and speed up the process of introgressing multiple determinants associated to desired traits.

In the context of the invention, double strand breaks are thus created on the chromosome(s) bearing the determinant(s). It may also be advantageous to create double strand breaks on the homologous chromosomes of the recurrent lines at the same loci than the ones of the determinant-bearing chromosomes, to further trigger chromosome recombination.

In the BC0 line (cross between a homozygous donor line bearing the trait and a homozygous recurrent line), some crossing over will occur during meiosis (FIG. 1). This will result in generation of gametes from the BC0 plants that will have the trait having moved from the donor line chromosome background to the recurrent line chromosomal background (FIG. 1.D).

After selection of the appropriate BC1 plants (from gametes (3) of FIG. 1.D) such BC1 plants are crossed with the recurrent line. The gametes obtained during meiosis of the BC1 plants will present a further recombination event in which the chromosomal background of the recurrent line (close to the telomere) will recombine so that the trait will be introgressed (gamete (2), FIG. 2. C).

One key feature of the tools herein disclosed is to force double-strand breaks during meiosis, through Spo11 action, at proper sites, at least on the donor line, in order to initiate recombination with the chromatid of the recurrent line.

It is thus necessary to target the Spo11 to the proper location on the chromatid.

The invention thus relates to a plant comprising
(a) a genetic determinant associated with a trait, wherein the genetic determinant is located on at least one chromosome of the plant, wherein the genetic determinant is positioned between two chromosomal regions I1.1 and I1.2, wherein each chromosomal region I1.1 and I1.2 is located between 1 cM and 5 cM of the genetic determinant,
(b) a genetic construct coding for a dead Cas protein
(c) at least a genetic construct allowing transcription of a guide RNA associated with the dead Cas protein, wherein the gRNA recognizes a DNA sequence located in the chromosomal region I1.1.
(d) a genetic construct coding for a Spo11 protein, wherein the Spo11 protein is fused to a component that allows recruitment of the Spo11 protein to the DNA sequence of the region I1.1 recognized by the guide RNA upon such recognition.

The plant may be homozygous for the genetic determinant (and hence contain two copies of the genetic determinant). This is favored as it would improve introgression of the trait. In some other embodiments, the plant is hemizygous for the genetic determinant (it contains only one copy of the genetic determinant).

This plant is the “donor” plant, containing the genetic determinant that is to be introgressed in the recurrent line.

The plant is able to express a guide RNA (gRNA) that can recognize a region I1.1 that is in the vicinity (within 1-5 cM, preferably at between 1 and 2 cM of the trait, or within the physical distance (10-1000 kb, preferably between 50-500 kb, or between 100-500 kb, more preferably between 50-100 kb) as indicated above), either between the centromere and the trait between the telomere and the trait.

Upon binding of the gRNA and of the dead Cas to this region, the Spo11 is recruited at this region and thus becomes able to induce a double-strand break.

The I1.1 region. which is targeted by the gRNA is preferably selected so that its sequence is as homologous as possible as the corresponding region of the recurring line, so that the gRNA can bind to the DNA of both the donor line and the recurring line.

However, in some embodiments, it is not possible to have such homology, and the plant also contains (e) a genetic construct allowing transcription of a guide RNA associated with the dead Cas protein, wherein the gRNA recognizes a DNA sequence located in the region of the chromosome of the recurrent line that corresponds to the chromosomal region I1.1.

The Spo11 protein is linked to a component (mean) that allows recruitment of the Spo11 protein to the DNA sequence of the region I1.1 recognized by the guide RNA upon recognition of the sequence by the gRNA.

It is possible to envisage multiple means to achieve this result:
i. in one embodiment, the Spo11 is fused to the dead Cas protein (the genetic constructs (b) and (d) are linked so that transcription and translation leads to production of a fusion dead Cas-Spo11 or Spo11-dead Cas protein. Upon binding of the complex gRNA-Cas protein to the region I1.1, the Spo11 is recruited to this region with the Cas protein. According to one embodiment, the Spo11 domain is on the N-terminal side and the dead Cas on the C-terminal side of the fusion protein. According to another embodiment, the Spo11 domain is on the C-terminal side and the dead Cas on the N-terminal side of the fusion protein.
ii. in another embodiment, the gRNA is fused to a RNA aptamer, and the Spo11 protein is produced in the form of a fusion protein with the target of the RNA aptamer. In this embodiment, upon binding of the complex gRNA-Cas protein to the region I1.1, the Spo11 is recruited to this region by binding of the gRNA-aptamer to the target bound to the Spo11 protein. As RNA aptamer, one can use an RNA aptamer derived from the operator stem-loop of bacteriophage MS2 (MS2) fused to the 3′ end of the gRNA scaffold with its ligand MS2 coat protein (MCP). In one embodiment, the genetic construct coding for the Spo11 protein also contains a sequence coding for a MS2 coat protein (MCP) so as to produce a fusion protein MCP-Spo 11, and the genetic construct allowing transcription of a guide RNA also contains a MS2 aptamer, so that the Spo11 protein is directed to the DNA sequence of the region I1.1 recognized by the guide RNA upon such recognition, through the binding of the MCP protein to the MAS2 aptamer.
iii. in another embodiment, the Spo11 protein is fused to a modular recognition domain (MRD), which recognizes the Cas protein. Hence, upon binding of the complex gRNA-Cas protein to the region I1.1, the Spo11 is recruited to this region through this MRD recognizing the Cas protein. As MRD, one can cite alternative scaffolds, based on VASP polypeptides, Avian pancreatic polypeptides (aPP), tetranectins (based on CTLD3), affitins (based on Sac7d of the hyperthermophilic archaeon), affilins (based on γB-crystallin/ubiquitin), knottins, SH3 domains (e.g., fynomers, see e.g. PCT publications WO 2008/022759 and WO 2011/02368), PDZ domains, tendamistat, transferrin, an ankyrin repeat consensus domains (e.g., DARPins), lipocalin protein folds (e.g., anticalins and duocalins), fibronectins (e.g., adnectins, as described in US 2003/0170753 and US 20090155275), knottins, Z-domain of protein A (e.g., affibodies), thioredoxin, albumin (e.g., ALBUdAb (Domantis/GSK), Kunitz type domains, ALB-Kunitz sequences (e.g., Dyax)), unstructured repeat sequences of 3 or 6 amino acids (e.g., PASylation® technology and XTEN® technology), centyrin scaffolding, and sequences containing elastin-like repeat domains.
iv. in another embodiment, the Spo11 protein is recruited to the target nucleotide region I1.1 (the region close to the determinant, or the corresponding region of the chromosomes of the recurring line) using any companion protein (DNA targeting protein) known in the art, which can be associated with the Spo11 and direct the Spo11 to the proper location in the genome. One can cite zinc-finger proteins, or TALEN.

It is preferred when the Spo11 protein is associated with an RNA-guided DNA endonuclease enzyme from the CRISPR-Cas system (however devoid of endonuclease activity, and hence called deadCas or dCas) to be provided to the target nucleotide sequence; i.e. when the Spo11 protein is fused to the dead Cas protein. In particular, the RNA-guided endonuclease from the CRISPR-Cas system is a type V CRISPR like Cas12a or a type II CRISPR like Cas9. Expression of an appropriate guide sequence (gRNA) will then target the fusion protein to the target DNA sequence I1.1. The plant may contain one gRNA targeting the I1.1 region or more than one gRNA (2 or 3) targeting different sequences of the I1.1 region.

It is thus preferred when a promoter that is active during meiosis is used to express the Spo11 and Cas proteins, or the fusion protein Cas-Spo11, as well as the guide RNAs. Such promoter can be meiosis specific (i.e. active only in the cells subject to meiosis, i.e. with no expression of a nucleic acid sequence encoding a protein operatively linked to it in other cells or in other conditions than meiosis), in order to prevent any performance of the system in cells that are not involved in the meiosis pathway. Alternatively, a constitutive promoter can be used, i.e. a promoter active in all cells, in particular in cells undergoing meiosis. Any promoter active in cells undergoing meiosis can be used. This can be easily determined by using a reporter gene (such as the gene coding for the GFP protein) and verifying expression of the protein in meiosis cells.

Examples of meiosis-specific promoters suitable for use in the present invention include, but are not limited to, endogenous Spo11 promoters, Spo11 partner promoters for double-strand break formation, the Rec8 promoter (Murakami & Nicolas, 2009, Mol. Cell. Biol, 29, pp. 3500-3516), or the Spo13 promoter (Malkova et al, 1996, Genetics, 143, pp. 741-754), meiotic promoters from Arabidopsis thaliana for example as described in Li et al., BMC Plant Biol. 2012; 12: pp. 104, Eid et al, Plant Cell Rep. 2016, 35(7) pp. 1555-1558, Xu et al, Front. Plant Sci, 13 Jul. 2018, Da Inès et al, PLoS Genet, 2013, 9, e1003787). One can also cite promoters described in WO2019224324.

Constitutive promoters suitable for use in the present invention include cytomegalovirus (CMV) early immediate gene promoter, simian virus (SV40) promoter, adenovirus major late promoter, Rous sarcoma virus (RSV) promoter, mouse mammary tumor virus (MMTV) promoter, phosphoglycerate kinase (PGK) promoter, EDI-alpha elongation factor promoter, ubiquitin promoters, actin promoters, tubulin promoters, immunoglobulin promoters, alcohol dehydrogenase 1 (ADH1) promoter, RNA polymerase III-dependent promoters such as U6, U3, HI, 7SL, pRPRI (“Ribonuclease P RNA 1”), SNR52 (“small nuclear RNA 52”), or the pZmUbi promoter (ubiquitin 1 promoter of maize (Christensen et al., 1996, Transgenic. Res., 5: 213)). Among constitutive promoters active in plants, one can cite the CsVMV promoter (Verdaguer et al, 1996, Plant Mol. Biol. 31, 1 129-39 and WO 97/48819), the rice actin promoter (McElroy et al., 1990, Plant Cell, 2: 163-171), the CaMV 35 S (35S promoter of the cauliflower mosaic virus) or the 19S promoter (Kay et al., 1987, Science, 236:1299-1302), the regulatory sequences of the T-DNA of Agrobacterium tumefaciens, including mannopine synthase, nopaline synthase, octopine synthase.

It is reminded that there are two major classes of CRISPR systems, according to the type of effector nuclease.

In the class 2 (or class II) system, the effector nuclease is a monomer and consists of a single polypeptide chain. The type II CRISPR systems have a Cas9 endonuclease, with two separate catalytic domains belonging to the RuvC and HNH catalytic groups as described above, with the HNH domain cutting the strand targeted by the guide (complementary to the guide) target strand, and the RuvC domain cutting the complementary strand of the strand targeted by the guide (non-complementary to the guide) non-target strand. The ability for Cas9 to cleave a DNA sequence depends on the presence of an adequate protospacer adjacent motif (PAM). When the Cas protein is a class 2 protein, the dead Cas protein can be a Cas9 that has a double mutation removing its ability to create a double strand DNA break at the target site (deadCas9-BE or dCas9-BE) such as the one disclosed in Komor et al (Nature 533, 420-424 (2016)). Indeed, the ability of Cas9 to create a double strand DNA break is mediated by two nuclease activities, a RuvC activity and an HNH activity. If one of these domains is mutated, the Cas9 enzyme loses its ability to cut the double-stranded DNA and can only cut one strand. Mutation D10A in Cas9 eliminates RuvC activity and H840A eliminates the HNH activity.

Type II Cas proteins (such as Cas9) cleave double-stranded DNA adjacent to protospacer adjacent motif (PAM) sequences.

Type V systems have a Cpf1, C2c1 or C2c3 type endonuclease. Type V Cas proteins (such as Cas12) cleave dsDNA adjacent to protospacer adjacent motif (PAM) sequences specifically and single-stranded DNA (ssDNA) nonspecifically. In this embodiment, the dead Cas protein is of type V and is the dead Cas12a protein. Such dead Cpf1 (notably Cas12a protein) can be obtained by introduction of one or several mutations in the RuvC domain, the only nuclease domain of Cas12a. The RuvC domain can be identified in any Cas12a by homology searches (Shmakov et al. 2015, Mol Cell. 60(3):385-97. doi: 10.1016/j.molcel.2015.10.008). The relevant positions to be mutated to inactivate the RuvC domains can also be identified by homology searches using known dead Cas12a.

As examples of mutations to obtain dead Cpf1, one can cite D908A, E993A, and D1263A in Acidaminococcus sp. BV3L6 (AsCpf1), D832A, E925A, D947A or D1180A in Lachnospiraceae bacterium ND 2006 (LbCpf1) and D917A or E1006A in Francisella novicida 0112 (FnCpf1).

In one embodiment, the dead Cas12a is a Cas12a modified at one or several of the following positions 832, 1006 and 1125 when aligned with LbCas12a (Cas12a protein from Lachnospiraceae bacterium). The preferred substitutions are D832A, E1006A, D1125A.

In one embodiment, the dead Cas12a is a LbCas12a modified at one or several of the following positions 832, 1006 and 1125. The preferred substitutions are D832A, E1006A, D1125A. By way of example, dLbCas12a (D832A) represented in SEQ ID NO: 83 of WO2021123397 and dLbCas12a (D832A/E1006A/D1125A) represented in SEQ ID NO: 8 of WO2021123397 are suitable

Alternatively, one can use a dead Cas12b protein as described by Ming et al. (Nat Plants. 2020 March; 6(3):202-208).

In some embodiments, the dead Cas protein or the fusion Spo11-dead Cas protein is associated with Nuclear Localization Signals (NLS) like the SV40 NLS (disclosed as SEQ ID NO: 88 of WO2021123397) or the XlNucleoplasmin NLS (disclosed as SEQ ID NO: 89 of WO2021123397). The fused protein can thus also include such NLS.

It is preferred when the Spo11 protein is part of a Cas Type II complex comprising a dead Type II Cas protein and the Spo11 protein. Such Cas Type II can be targeted to the sequence of interest by an appropriate guide RNA (gRNA) associated with the dead Type II Cas protein, which can herein be described as a first guide RNA, and promote meiotic recombination by way of the Spo11 protein. The dead Cas Type II protein is preferentially a dead Cas9.

When driving the Spo11 protein to the desired DNA location(s) using a protein of the CRISPR-Cas system, one shall also use sets of guides RNA tailored/optimized for the trait introgression (i.e. targeting the genome located within 1-5 cM, preferably at between 1 and 2 cM of the genetic determinant associated with the trait). It may also be located within the physical distance (10-1000 kb, preferably between 50-500 kb, or between 100-500 kb, more preferably between 50-100 kb) as indicated above. The guide RNA can be designed to specifically target the selected regions of homology between the donor and recurrent lines that flank the donor region to be introgressed into the recurrent line (recipient line in which the genetic determinant is to be introgressed).

The guides may target only the sequence of the donor line or only the sequence of the recurrent line or can target both the sequences of the donor and the recurrent lines. The gRNA can be designed to be functional on a large number of recurrent lines by leveraging the set of available genomic sequences at the locus of the regions to be introgressed. Hence, the most conserved part of the target regions can be identified and selected for gRNA design. gRNAs are then designed according to the specific CRISPR nuclease that is used, including the PAM and folding requirements. Thereafter, the specificity of the gRNA is checked in a step of genome-wide homology search. Specific gRNAs for the targeted loci are retained for the final construct design.

When used for introgressing transgenes (GM events), i.e. when the determinant is a transgene, the technology could be entirely embedded in the GM donor line.

The donor line shall thus contain the transgene coding for the DNA-targeting protein-Spo11 fusion protein and the set of guides RNA (if appropriate) tailored/optimized for the trait introgression (which depend on the location of the transgene (GM event) in the genome of the donor line).

In one embodiment, the plant comprises another genetic construct allowing transcription of a guide RNA associated with the dead Cas protein, wherein the gRNA recognizes a DNA sequence located in the chromosomal region I1.2, and wherein the Spo11 protein is directed to the DNA sequence of the region I1.2 recognized by the guide RNA upon such recognition.

The purpose of this other gRNA is to target the Spo11 protein also in the I1.2 region, so that a DSB occurs both in the I1.1 and I1.2 regions. Since DSB induced by Spo11 triggers recombination, forcing two DSB around a specific determinant should increase the frequency of recombination.

The constructs are of particular interest when multiple determinants associated with different traits are to be introgressed from the donor line within the recurrent line.

In particular, when two traits are to be present in the recurrent line, the plant comprises a second genetic determinant associated with a trait (generally another trait than the trait associated with the first genetic determinant located between I1.1 and I1.2).

The plant may be homozygous for the second genetic determinant (and hence contain two copies of the second genetic determinant) or hemizygous for the second genetic determinant (it contains only one copy of the second genetic determinant).

The second genetic determinant is positioned between two chromosomal regions I2.1 and I2.2. Each chromosomal region I2.1 and I2.2 is located between 1 cM and 5 cM (preferably between 1 and 2 cM) of the second genetic determinant. It may also be located within the physical distance (10-1000 kb, preferably between 50-500 kb, or between 100-500 kb, more preferably between 50-100 kb) as indicated above.

In a preferred embodiment, the chromosome bearing the second genetic determinant is different from the chromosome bearing the first genetic determinant. In another embodiment, the second genetic determinant is on the same chromosome that the one bearing the first genetic determinant, but the two genetic determinants are located on different sides from the centromere of the chromosome. It is indeed preferable not to induce too many recombination events on the same arm of a chromosome.

The plant also comprises at least a genetic construct allowing transcription of a guide RNA associated with the dead Cas protein, wherein the gRNA recognizes a DNA sequence located in the chromosomal region I2.1.

The Spo11 protein has to be directed to the DNA sequence of the region I2.1 recognized by the guide RNA upon such recognition.

In view of the fact that the Spo11 protein should operate at the I1.1 and I2.1 (and optionally at the I1.2) regions, it is advisable when the means allowing Spo11 to go to these regions are identical for each gRNA. Consequently, it is preferred to use the fusion Spo11-dead Cas protein, or to use a fusion aptamer target-Spo11 protein, with all guides RNA also containing the same RNA aptamer.

In one embodiment, the plant also comprises another genetic construct allowing transcription of another guide RNA associated with the dead Cas protein, wherein the other guide RNA recognizes a DNA sequence located in the chromosomal region I2.2, and wherein the Spo11 protein is directed to the DNA sequence of the region I2.2 recognized by this other guide RNA upon such recognition.

In another embodiment, the plant can be used for introgressing three traits.

In this embodiment, the plant comprises a third genetic determinant associated with a trait, located on at least one chromosome of the plant, and positioned between two chromosomal regions I3.1 and I3.2, wherein each chromosomal region I3.1 and I3.2 is located between 1 cM and 5 cM (preferably between 1 and 2 cM) of the third genetic determinant. It may also be located within the physical distance (10-1000 kb, preferably between 50-500 kb, or between 100-500 kb, more preferably between 50-100 kb) as indicated above. In this embodiment, it is preferred when the third determinant is not located on the same chromosome arm as the first or second determinant.

The plant may be homozygous for the third genetic determinant (and hence contain two copies of the third genetic determinant) or hemizygous for the third genetic determinant (it contains only one copy of the third genetic determinant).

The plant also comprises at least a genetic construct allowing transcription of a guide RNA associated with the dead Cas protein, wherein the gRNA recognizes a DNA sequence located in the chromosomal region I3.1, wherein the Spo11 protein is directed to the DNA sequence of the region I3.1 recognized by the guide RNA upon such recognition. Optionally, the plant also comprises another genetic construct allowing transcription of another guide RNA associated with the dead Cas protein, wherein the other guide RNA recognizes a DNA sequence located in the chromosomal region I3.2, wherein the Spo11 protein is directed to the DNA sequence of the region I3.2 recognized by the other guide RNA upon such recognition.

In another embodiment, the plant can be used for introgressing four traits.

In this case, the plant further comprises a fourth genetic determinant associated with a trait, located on at least one chromosome of the plant, and positioned between two chromosomal regions I4.1 and I4.2, wherein each chromosomal region I4.1 and I4.2 is located between 1 cM and 5 cM (preferably between 1 and 2 cM) of the fourth genetic determinant. It may also be located within the physical distance (10-1000 kb, preferably between 50-500 kb, or between 100-500 kb, more preferably between 50-100 kb) as indicated above.

The plant may be homozygous for the fourth genetic determinant (and hence contain two copies of the fourth genetic determinant) or hemizygous for the fourth genetic determinant (it contains only one copy of the fourth genetic determinant).

It is preferred when the fourth genetic determinant is not located on the same chromosome arm as the first, second or third determinant.

The plant further comprises at least a genetic construct allowing transcription of a guide RNA associated with the dead Cas protein, wherein the gRNA recognizes a DNA sequence located in the chromosomal region I4.1, wherein the Spo11 protein is directed to the DNA sequence of the region I4.1 recognized by the guide RNA upon such recognition.

Optionally, the plant also comprises another genetic construct allowing transcription of another guide RNA associated with the dead Cas protein, wherein the other guide RNA recognizes a DNA sequence located in the chromosomal region I4.2, wherein the Spo11 protein is directed to the DNA sequence of the region I4.2 recognized by the other guide RNA upon such recognition.

In another embodiment, the plant can be used for introgressing five traits.

In this case, the plant comprises a fifth genetic determinant associated with a trait, located on at least one chromosome of the plant, and positioned between two chromosomal regions I5.1 and I5.2, wherein each chromosomal region I5.1 and I5.2 is located between 1 cM and 5 cM (preferably between 1 and 2 cM) of the fifth genetic determinant. It may also be located within the physical distance (10-1000 kb, preferably between 50-500 kb, or between 100-500 kb, more preferably between 50-100 kb) as indicated above.

The plant may be homozygous for the fifth genetic determinant (and hence contain two copies of the fifth genetic determinant) or hemizygous for the fifth genetic determinant (it contains only one copy of the fifth genetic determinant).

It is preferred when the fifth genetic determinant is not located on the same chromosome arm as the first, second, third and fourth determinant.

The plant then further comprises at least a genetic construct allowing transcription of a guide RNA associated with the dead Cas protein, wherein the gRNA recognizes a DNA sequence located in the chromosomal region I5.1, wherein the Spo11 protein is directed to the DNA sequence of the region I5.1 recognized by the guide RNA upon such recognition. Optionally, the plant also comprises another genetic construct allowing transcription of another guide RNA associated with the dead Cas protein, wherein the other guide RNA recognizes a DNA sequence located in the chromosomal region I5.2, wherein the Spo11 protein is directed to the DNA sequence of the region I5.2 recognized by the other guide RNA upon such recognition.

The invention also relates to a cell of a plant containing the elements mentioned above.

It is preferred when the various elements allowing occurrence of the DSB near the genetic determinant(s) (namely the dead Cas, Spo11 proteins and gRNAs) are located at the same locus. Preferably, the locus is not on the same arm of a chromosome bearing the genetic determinant(s) associated to the trait(s) to introgress.

Using plants, containing the determinant and the construct (DNA-targeting protein/Spo11, gRNA) herein disclosed, in processes of trait introgression would provide the following advantages:
Example for trait introgression of only one trait: the gene containing the DNA-targeting protein fused with the Spo11 protein would be inserted in the donor plant genome which means that 100% of the F1 (BC0) plants have the technology (the determinant, the dead Cas, one or more gRNA and the Spo11 protein, potentially fused to the Cas protein). Consequently, 50% of the gametes produced by such plant contain the trait. Assuming that the efficacy of the technology is 100%, 50% of the BC1 seeds would be double recombinant, at the left and right borders of the genetic determinant for the targeted trait (when using two gRNA).
Simultaneous introgression of 2 traits: 25% of the gametes produced from the BC0 plants will contain the 2 traits of interest. With a technology efficacy of 100%, 25 plants should be sufficient to identify the perfect targeted plants with the 4 recombination events, i.e. at each border of the 2 traits (with a probability above 99%).
For introgressing 5 traits simultaneously: 3% of the gametes produced from the BC0 plants contain the 5 traits. With a technology efficacy of 100%, 200 plants should be sufficient to identify the perfect targeted plants with the 10 recombination events (right and left border of each trait).

The efficacy of the technology may not be 100%, as it is expected that there might be some crossing-over interference, between the two borders of the same trait, which could require production of some recombinant borders at BC1 meiosis and complementary one at BC2 meiosis.

More generally it is preferred to identify, in the BC1 seeds, the seeds for which:
the more genetic determinants have recombined at the left and right border and are introduced within the recurrent line genome, and
   If all traits have recombined at the left and right borders,
      the genome ratio for the recurrent line is the highest and
      the BC1 seed doesn't contain the technology, as there is no further need to trigger recombination for the traits
   If recombination for some traits has not occurred at the two borders,
      the traits which have recombined at the two borders are present,
      the other traits (which have not recombined at the two borders) are also present,
      the genome ratio for the recurrent line is the highest and
      the BC1 seed contains the technology (machinery for generating the DSB as disclosed), as it will be required to operate during meiosis preparing the gametes that will lead to the BC2 plants.

One advantage to use a deadCas-Spo11 fusion protein (in particular a deadCas9-Spo11) is that it is possible to use one single DNA-targeting protein, and design several guides RNA (gRNA) (from 3 to 5, well scattered in the interval of 1 cM or 2 cM (or for example for Corn within one to few megabases before and after the genetic determinant associated with the trait)) on each border (right & left) of each genetic determinant to introgress.

Chances of having a crossing-over in such locations are thus increased as this strategy:
Maximizes the number of Spo11-induced double strand DNA breaks (DSB) in that sub-region versus the ones occurring distantly,
Ensures that some DSB arise, even if, in the microenvironment, there is no perfect homology between the donor and recurrent parents (it is reminded that DSB would arise naturally after that two homologous chromosomal regions match). This is particularly true when there is need to introgress non-conventional genetic determinants (for instance specific transgenes (GM events), or determinants that come from plant varieties that are genetically distant from the elite line that need to receive the genetic determinant (designated as exotic introgressions)).
Decreases the risk of recombination inhibition by epigenetic specific microenvironment in some breeding lines, as the DSB is forced by the machinery/technology herein disclosed.

A typical genetic construct for ensuring good frequency recombination on each side of 5 different Traits scattered on different chromosomes, may have:
A transgene coding for a dead Cas9-Spo11 (or another deadCas-Spo11 protein) fusion protein under control of a promoter active during meiosis (such as a Spo11 promoter) or of a strong constitutive promoter
12 to 25 cassettes driving transcription of the gRNAs in particular under control of an RNA Pol III (RNA polymerase III) promoter, in particular during meiosis.

Alternatively, such a set of gRNA can also be directly multiplexed, under the transcriptional control of an appropriate promoter, such as an RNA Pol II promoter or RNA Pol III promoter, or a meiosis or constitutive promoter, in particular as listed above, in a polycistronic-like transcript where each gRNA is for example flanked by ribozyme sequence to allow for processing into individualized guides RNA (He et al. J Genet Genomics. 2017 Sep. 20; 44(9): 469-472). Other multiplexing strategies include the use of tRNAs or Csy4 to separate the guides.

It may be needed (and is advised) to use several “multiplexed” cassettes with less than a dozen of gRNAs in each. This will ensure good productivity of the system, in view of the significant number of gRNAs.

The genetic construct may also contain
A selective marker (antibiotic or herbicide tolerance gene) to produce the initial transgenic recombination-inducing line.

The multimerization of guides for each genetic determinant associated with a trait side is a good way to maximize efficiency of the process and is thus preferred. Nevertheless, it is not mandatory and the invention might also be performed with only one or two guides.

It is also envisaged to transiently deliver guides RNAs in a transgenic material where a DeadCas-Spo11 fusion protein is expressed.

The invention also relates to a method for obtaining a plant comprising a genetic determinant associated with a trait, wherein the genetic determinant is located on at least one chromosome of the plant, wherein the genetic determinant is positioned between two chromosomal regions I1.1 and I1.2, wherein each chromosomal region I1.1 and I1.2 is located between 1 cM and 5 cM of the genetic determinant (preferably between 1 and 2 cM) (It may also be located within the physical distance (10-1000 kb, preferably between 50-500 kb, or between 100-500 kb, more preferably between 50-100 kb) as indicated above), comprising introducing, in a plant comprising the genetic determinant, one or more transgene(s) comprising
a genetic construct coding for a dead Cas protein
a genetic construct allowing transcription of at least one guide RNA associated with the dead Cas protein, wherein the gRNA recognizes a DNA sequence located in the chromosomal region I1.1,
a genetic construct coding for a Spo11 protein, wherein the Spo11 protein is fused to means that makes the Spo11 protein being directed to the DNA sequence of the region I1.1 recognized by the guide RNA upon such recognition.

It is preferred when the genes coding for the dead Cas protein and the Spo11 protein are under control of a promoter active during meiosis prophase I.

It is desirable that the gRNA is transcribed during meiosis prophase I.

As indicated above, one prefers when the means that makes the Spo11 protein being directed to the DNA sequence of the region I1.1 recognized by the guide RNA upon such recognition consists in the fusion of the Spo11 protein to the dead Cas protein.

In the preferred embodiment, the dead Cas protein is a dead Cas9 protein.

The invention also relates to a method for obtaining a plant comprising a determinant associated with a trait, comprising introducing, in a plant comprising the genetic determinant, one or more transgene(s) comprising
A genetic construct coding for a dead Cas protein under control of a promoter expressed during meiosis or of a constitutive promoter,
A genetic construct coding for a Spo11 protein,
cassettes driving transcription of gRNAs in particular under control of a RNA Pol III promoter, wherein the gRNAs are guide RNAs associated with the dead Type II Cas protein and target the dead Type II Cas protein to genomic regions located within 2 cM of the genetic determinant.

The genetic determinant is to be introgressed in a recurrent line. Preferably, the plant contains more than one genetic determinant (two, three, four or even five genetic determinants).

The genetic constructs are introduced in the donor plant. The donor plant can be a monocotyledonous or a dicotyledonous plant.

A monocotyledonous plant in which the technology is to be implemented can be a wheat plant, a corn plant, a maize plant, a rice plant, a barley plant, an oat plant, a sorghum plant.

A dicotyledonous plant in which the technology is implemented can be a cotton plant, a soybean plant, a beet plant, a potato plant, a tomato plant, a Brassica plant.

Preferably the plant cell is selected from monocotyledonous and dicotyledonous plants, more preferably selected from the group consisting of rice, wheat, soybean, corn, tomato, onion, cucumber, lettuce, asparagus, carrot, turnip, Arabidopsis thaliana, barley, rapeseed, cotton, grapevine, sugar cane, beet, cotton, sunflower, oil palm, coffee, tea, cocoa, chicory, bell pepper, chilli, lemon, orange, nectarine, mango, apple, banana, peach, apricot, sweet potato, yams, almond, hazelnut, strawberry, melon, watermelon, olive, potato, zucchini, eggplant, avocado, cabbage, plum, cherry, pineapple, spinach, apple, tangerine, grapefruit, pear, grape, clove, cashew, coconut, sesame, rye, hemp, tobacco, berries such as raspberry or blackcurrant, peanut, castor, vanilla, poplar, eucalyptus, green foxtail, cassava and horticultural plants such as roses, tulips, orchids and geraniums. In particular, the plant cell can be selected from the group consisting of rice, wheat, soybean, corn, tomato, onion, cucumber, lettuce, asparagus, carrot, turnip, Arabidopsis thaliana, barley, rapeseed, cotton, grapevine, sugarcane, beet, cotton, sunflower, palm olive, coffee tea, cocoa, chicory, bell pepper, chili, lemon, orange, nectarine, mango, apple, banana, peach, apricot, sweet potato, yams, almonds, hazelnuts, strawberries, melons, watermelons, olives, and horticultural plants such as roses, tulips, orchids and geraniums.

The donor plant for the trait(s) to introgress comprises the genetic construct as described herein.

In summary, is herein disclosed a method for introducing a genetic determinant associated with a trait in a recipient chromosome in a plant, comprising
(a) Crossing a first plant (donor) comprising the genetic determinant associated with the trait on a chromosome homologous to the recipient chromosome, with a second plant (recurrent parent)
   Wherein the first plant also comprises, in its genome a genetic construct coding for a fusion protein comprising a DNA-targeting protein fused with a Spo11 protein, with elements allowing expression of the protein at least during meiosis and targeting of the Spo11 protein within 2 cM of the genetic determinant (it may also be located within the physical distance (10-1000 kb, preferably between 50-500 kb, or between 100-500 kb, more preferably between 50-100 kb) as indicated above)
(b) Performing n backcrosses with the recurrent parent to obtain BCn plants
(c) Identifying BCn plants having the highest recurrent parent recovering percentage and having integrated the genetic determinant associated with the trait in the recipient chromosome.

As indicated above, the system in the first plant is preferably dead Cas-Spo11 fusion protein, and the gRNA as disclosed are also present in the first plant (which is the donor plant).

In summary, is herein disclosed a method for introducing a genetic determinant associated with a trait in a recipient chromosome in a plant, comprising
(a) Crossing a first plant (donor) comprising the genetic determinant associated with the trait on a chromosome homologous to the recipient chromosome, with a second plant (recurrent parent)
   Wherein the first plant also comprises, in its genome a genetic construct coding for a fusion protein comprising a DNA-targeting protein fused with a Spo11 protein, with elements allowing expression of the protein at least during meiosis and targeting of the Spo11 protein within 1-5 cM (preferably about 2 cM) of the genetic determinant (it may also be located within the physical distance (10-1000 kb, preferably between 50-500 kb, or between 100-500 kb, more preferably between 50-100 kb) as indicated above)
(b) So as to obtain BC0 plants
(c) Repeating n times
   Performing a backcross with the recurrent parent to obtain BCn plants, wherein the backcross is performed with the plants from the previous backcross selected as having the highest recurrent parent recovering percentage and having integrated the genetic determinant associated with the trait in the recipient chromosome.

(d) After n backcrosses, identifying BCn plants having the highest recurrent parent recovering percentage and having integrated the genetic determinant associated with the trait in the recipient chromosome.

In these methods, due to the efficiency of the technology to introgress the trait, and in particular to introgress multiple traits, even though multiple backcrosses may be needed to get rid of the donor line genome, the need for screening will be reduced to the BC1 and BC2 plants.

These methods make it possible to understand the invention and are also part of the invention.

It is preferred when the DNA-targeting protein is a dead Type II Cas protein, in particular a dead Cas9 protein. In this embodiment, the first plant also comprises cassettes driving transcription of gRNAs, wherein the gRNAs are guide RNAs associated with the dead Type II Cas protein so that the dead Type II Cas protein (associated with the Spo11 protein) is targeted to regions within 2 cM of the a genetic determinant associated with the trait to be introgressed. In some embodiments, the genetic construct coding for the fusion protein comprising the DNA-targeting protein fused with the Spo11 protein, and the cassettes driving transcription of gRNAs (if needed) are not on the same chromosome than the genetic determinant associated with the trait. In another embodiment, the genetic construct coding for the fusion protein comprising the DNA-targeting protein fused with the Spo11 protein, and the cassettes driving transcription of gRNAs (if needed) are on the same chromosome.

In some embodiments, 2 backcrosses are performed (n=2, BC2 plants are obtained). In some embodiments, 3 backcrosses are performed (n=3, BC3 are obtained). In some embodiments, 4 backcrosses are performed (n=4, BC4 plants are obtained). It is also envisaged that more than 4 backcrosses are performed n>4), although this embodiment is not frequent.

In some embodiments, the second backcross (to obtain the BC2 plants) is performed on BC1 plants which are selected to also contain the genetic construct coding for the fusion protein comprising the DNA-targeting protein fused with a Spo11 protein.

It is reminded that a backcross consists in crossing plants from progeny of the previous cross with the recurrent parent. The plants that are crossed are the plants of the progeny of the previous cross that present the trait of interest, and the highest recurrent parent recovering percentage (i.e. the highest percentage of markers that belong to the recurrent parent). Such plants that are used in the successive backcrosses can be selected, after each backcross, by methods known in the art and preferably by use of appropriate genetic markers.

There is also disclosed a method for obtaining a donor plant (i.e. a plant that comprises a genetic determinant associated with a trait to be introgressed in a recurrent line), comprising
Introducing, in a plant comprising the genetic determinant associated with the trait to be introgressed in the recurrent line, one or more transgene(s) comprising
   A genetic construct coding for a dead Type II Cas (in particular a dead Cas9)-Spo11 fusion protein under control of a promoter expressed during meiosis or of a constitutive promoter
   cassettes driving transcription of the gRNAs in particular under control of a RNA Pol III promoter, wherein the gRNAs are guide RNAs associated with the dead Type II Cas protein and target the dead Type II Cas protein to regions within 2 cM of the genetic determinant associated with the trait to be introgressed.

It is possible to introduce the various elements listed above as a unique transgene, or in different transgenes (one transgene with the genetic construct coding for a dead Type II Cas-Spo11 fusion protein and one or more transgene(s) comprising the cassettes driving transcription of the gRNAs).

The donor plant can comprise one, two, three, four, five, more than five genetic determinants associated with trait(s) to be introgressed in a recurrent line.

This method makes it possible to understand the invention and is also part of the invention.

An object of the invention is also a plant (donor plant) that comprises a genetic determinant associated with a trait to be introgressed in a recurrent line, and comprises as transgenes, one genetic construct coding for a dead Cas type II protein, one genetic construct coding for a Spo11 protein (it is to be noted that the two proteins could be encoded by the same genetic construct so as to produce a fusion protein as disclosed herein), cassettes driving transcription of gRNAs, wherein the gRNAs are guide RNAs associated with the dead Type II Cas protein and target the dead Type II Cas protein to regions within 2 cM of traits to be introgressed.

It is also disclosed a method for detecting insertion of a genetic determinant associated with a trait in a recipient chromosome, comprising
(a) Identifying, in a population of plants, the plants that carry the genetic determinant associated with the trait flanked by sequences of the recipient chromosome

Wherein the population of plants has been obtained by the method herein disclosed, and wherein the plants are the recipient plants.

Such method can be performed by techniques known in the art, in particular using genetic markers. One can indeed design genetic markers that are specific of the recipient chromosomes. This method makes it possible to understand the invention and is also part of the invention.

Advantages of the Technology and Comments
genotyping 10 000 seeds at BC1 and BC2 is no more an issue with the genotyping technology that are currently available, thus increasing ability to identify multiple seeds with recombination events, such volumes could be needed depending on the efficiency of the technology.
obtaining multiple seeds with recombination events will improve the recurrent parent recovering percentage: there will be more seeds to screen and to choose from, with contain the highest recurrent parent genome ratio. This will reduce time to deliver the final product to the market, and increase the elite line genetic material therein. Indeed, there will be simplification of the final evaluation process as the converted line and the recurrent breeding line will have a very close genetic distance.
since the technology is preferably embedded in the donor genome, this application is easy to deploy. The donor plant with all traits of interest and the techno can easily be obtained (by crossing a transgenic plant with a plant containing the technology). This provides also opportunity for valorizing the plant containing, within its genome, the DNA-targeting protein-Spo11 fusion genetic construct.
Using one guide per side of the genetic determinant associated with a trait may be used in order to avoid a double crossing over (that would be counter-productive), in particular depending on the efficiency of the process and absence of crossing-over interference.
Better control the nature and size of the genomic fragment containing the trait from the donor line that is introgressed within the recurrent line, rather than relying on a random natural integration.
   in summary, the technology, methods, plants and genetic constructs described there-above make it possible to speed up introgression processes and deliver commercial and elite product to the market in a competitive time.

### DESCRIPTION OF THE FIGURES

FIG. 1: schematic representation of the crossing-over process during meiosis I. Light gray: chromosome of the donor line. Dark gray: chromosome of the recurrent (receiving) line. Black line: trait of interest. A. after replication, sister chromatids of the donor line and of the recurrent line come close together. B. exchange between chromosomal regions of the donor and recurrent lines. C. resulting chromosomes, with the trait being introduced in the recurrent line. D. The fours gametes (1) to (4) produces after meiosis II. The progeny carrying chromosome (3) will be selected for future backcross.

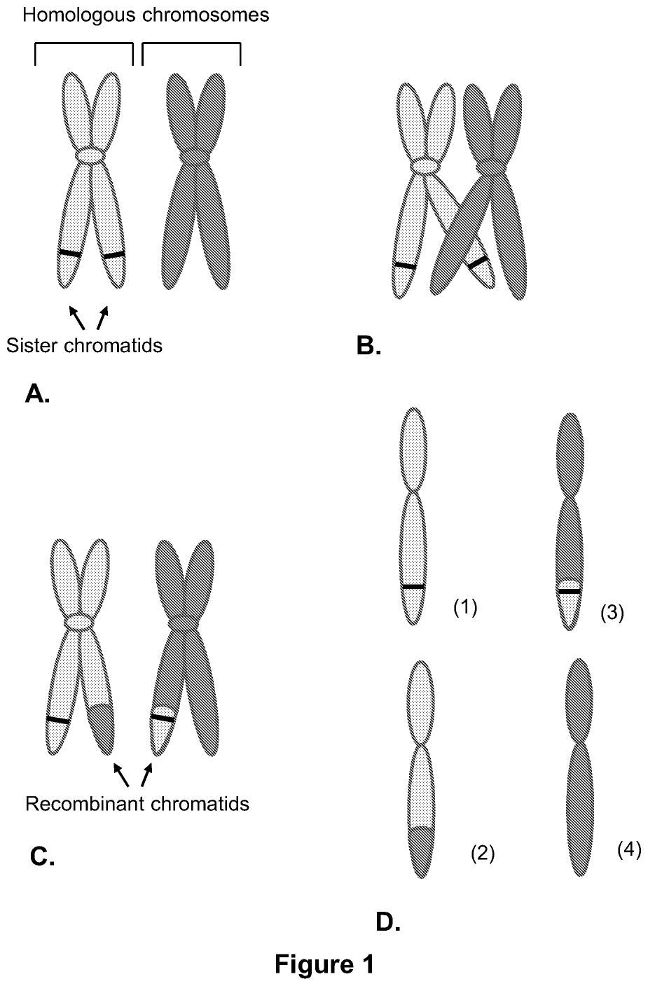

FIG. 2: schematic representation of the crossing-over process during meiosis I (prophase), for the progeny selected from FIG. 1 and the recurrent line. A. after replication, sister chromatids of the progeny (left) and of the recurrent line (right) come close together. B. Resulting chromosomes, with the distal arm of the recurrent line chromosome being introduced in the chromosome from the progeny. C. Resulting gametes where the trait has been introgressed in the genetic background of the recurrent line. Plants carrying cells issued from gamete (2) will be selected.

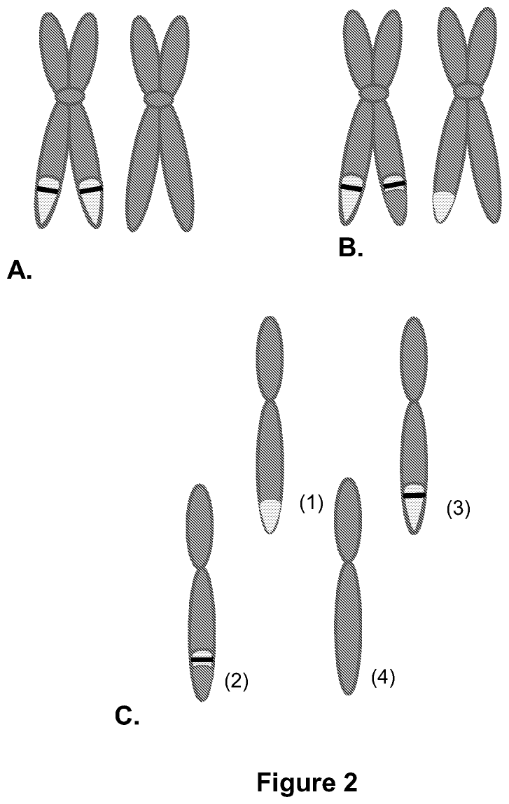

FIG. 3. Schematic representation of the trait introgression process. The donor line is crossed with the recurrent line and the BC0 plants containing the trait and the best genome ratio for the recurrent line are selected and crossed with the recurrent line. BC1 plants are selected and the process is iterated until the trait introgression is completed.

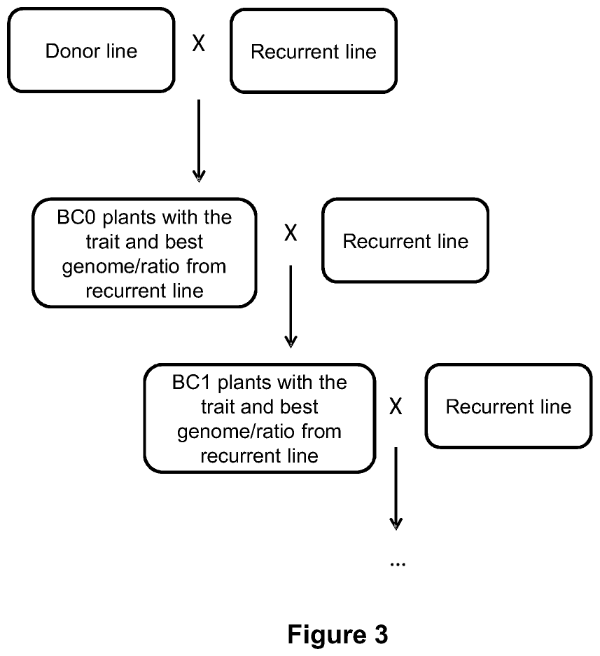

### EXAMPLES

### Example on Current GM Traits Introgression in Corn

As an illustration, the list of traits that can be stacked in a single donor carrying the targeted recombination technology are:

TABLE 1
Localization of genetic determinants in a maize donor

| | | Position on | Physical |
|---|---|---|---|
| GM Trait name | Chr | genetic map | position B73 V4 |
| 5307 (SYN-05307-1) | 5 | ~189.8 | ~92914987 |
| 3272 (SYN-E3272-5) | 6 | ~34.4 | ~31276706 |
| MIR604 | 1 | ~196.4 | ~39357162 |
| MON810 | 5 | ~170.7 | ~55833733 |
| MON88017 | 4 | ~171 | ~160587847 |
| DAS-59122 | 1 | ~502.1 | ~276806012 |

One can take as a border, a sequence that is at 500 kb or less from the insertion (genetic determinant), the distance being selected to ensure that the infrastructure of the locus is not impacted by the crossing over, and with regards to the homology of the selected sequence between the donor and recurrent lines.
In one embodiment, the left border is between −100-500 kb (the sign “−” indicates that the left border is between the centromere and the determinant).
In one embodiment the left border can be between −1.5 mbp and −0.5 mbp from the introgression. It can also be closer, between 100-500 kb or less.
In another embodiment, the left border can be between −2.5 and −1.5 mbp
In another embodiment, the left border can be between −3.5 and −2.5 mbp
   The right border can be between +100-500 kb ((the sign “+” indicates that the right border is between the determinant and the telomere), between +0.5 mbp and +1.5 mbp or between +1.5 mbp and +2.5 mbp or between +2.5 mbp and +3.5 mbp from the introgression of the genetic determinant.
   gRNAs are designed to target the borders, and a cassette/vector containing the gRNA sequences, and a gene coding for a fusion protein dead Cas9-Spo11 is introduced in the single donor by transgenesis.
   Plants are regenerated and made homozygous for the genetic determinants and the transgene coding for the crossing-over machinery.
   The homozygous plant is crossed with a recurrent line to obtain BC0 plants.
   The BC0 plants are crossed with the recurrent line to obtain BC1 plants. The BC1 plants are screened to identify the ones in which recombination has occurred, leading to introduction of the genetic determinants within the recurrent line chromosome (introduction of the genetic determinants within the recurrent line genomic background).
   Another cross of BC1 plants with the recurrent line can be needed to improve introgression.

1. A plant comprising, within its genome,
(a) a genetic determinant associated with a trait, wherein the genetic determinant is located on at least one chromosome of the plant, wherein the genetic determinant is positioned between two chromosomal regions I1.1 and I1.2, wherein each chromosomal region I1.1 and I1.2 is located between 1 cM and 5 cM of the genetic determinant,
(b) a genetic construct coding for a dead Cas protein,
(c) at least a genetic construct allowing transcription of a guide RNA associated with the dead Cas protein, wherein the gRNA recognizes a DNA sequence located in the chromosomal region I1.1,
(d) a genetic construct coding for a Spo11 protein, wherein the Spo11 protein is fused such that the Spo11 protein is directed to the DNA sequence of the region I1.1 recognized by the guide RNA upon such recognition.

2. The plant of claim 1, wherein the genetic construct coding for the dead Cas protein is fused to the genetic construct coding for the Spo11 protein to produce a fusion protein Cas-Spo11, and wherein the Spo11 protein is directed to the DNA sequence of the region I1.1 recognized by the guide RNA upon such recognition, through the binding of the dead Cas protein to the guide RNA and DNA sequence.

3. The plant of claim 1, wherein the genetic construct coding for the Spo11 protein comprises a sequence coding for a MS2 coat protein (MCP) to produce a fusion protein MCP-Spo 11, and wherein the genetic construct allowing transcription of a guide RNA also comprises a MS2 aptamer, so that the Spo11 protein is directed to the DNA sequence of the region I1.1 recognized by the guide RNA upon such recognition, through the binding of the MCP protein to the MAS2 aptamer.

4. The plant of claim 1, further comprising a genetic construct allowing transcription of another guide RNA associated with the dead Cas protein, wherein the other gRNA recognizes a DNA sequence located in the chromosomal region I1.2, and wherein the Spo11 protein is directed to the DNA sequence of the region I1.2 recognized by the guide RNA upon such recognition.

5. The plant of claim 1, further comprising a second genetic determinant associated with a trait, located on at least one chromosome of the plant, and positioned between two chromosomal regions I2.1 and I2.2, wherein each chromosomal region I2.1 and I2.2 is located between 1 cM and 5 cM of the second genetic determinant, wherein the plant further comprises at least a genetic construct allowing transcription of a second guide RNA associated with the dead Cas protein, wherein the gRNA recognizes a DNA sequence located in the chromosomal region I2.1, wherein the Spo11 protein is directed to the DNA sequence of the region I2.1 recognized by the guide RNA upon such recognition, and optionally another genetic construct allowing transcription of another guide RNA associated with the dead Cas protein, wherein the other guide RNA recognizes a DNA sequence located in the chromosomal region I2.2, wherein the Spo11 protein is directed to the DNA sequence of the region I2.2 recognized by the other guide RNA upon such recognition.

6. The plant of claim 5, further comprising a third genetic determinant associated with a trait, located on at least one chromosome of the plant, and positioned between two chromosomal regions I3.1 and I3.2, wherein each chromosomal region I3.1 and I3.2 is located between 1 cM and 5 cM of the third genetic determinant, wherein the plant further comprises at least a genetic construct allowing transcription of a third guide RNA associated with the dead Cas protein, wherein the gRNA recognizes a DNA sequence located in the chromosomal region I3.1, wherein the Spo11 protein is directed to the DNA sequence of the region I3.1 recognized by the guide RNA upon such recognition, and optionally another genetic construct allowing transcription of another guide RNA associated with the dead Cas protein, wherein the other guide RNA recognizes a DNA sequence located in the chromosomal region I3.2, wherein the Spo11 protein is directed to the DNA sequence of the region I3.2 recognized by the other guide RNA upon such recognition.

7. The plant of claim 6, further comprising a fourth genetic determinant associated with a trait, located on at least one chromosome of the plant, and positioned between two chromosomal regions I4.1 and I4.2, wherein each chromosomal region I4.1 and I4.2 is located between 1 cM and 5 cM of the fourth genetic determinant, wherein the plant further comprises at least a genetic construct allowing transcription of a fourth guide RNA associated with the dead Cas protein, wherein the gRNA recognizes a DNA sequence located in the chromosomal region I4.1, wherein the Spo11 protein is directed to the DNA sequence of the region I4.1 recognized by the guide RNA upon such recognition, and optionally another genetic construct allowing transcription of another guide RNA associated with the dead Cas protein, wherein the other guide RNA recognizes a DNA sequence located in the chromosomal region I4.2, wherein the Spo11 protein is directed to the DNA sequence of the region I4.2 recognized by the other guide RNA upon such recognition.

8. The plant of claim 7, further comprising a fifth genetic determinant associated with a trait, located on at least one chromosome of the plant, and positioned between two chromosomal regions I5.1 and I5.2, wherein each chromosomal region I5.1 and I5.2 is located between 1 cM and 5 cM of the fifth genetic determinant, wherein the plant further comprises at least a genetic construct allowing transcription of a fifth guide RNA associated with the dead Cas protein, wherein the gRNA recognizes a DNA sequence located in the chromosomal region I5.1, wherein the Spo11 protein is directed to the DNA sequence of the region I5.1 recognized by the guide RNA upon such recognition, and optionally another genetic construct allowing transcription of another guide RNA associated with the dead Cas protein, wherein the other guide RNA recognizes a DNA sequence located in the chromosomal region I5.2, wherein the Spo11 protein is directed to the DNA sequence of the region I5.2 recognized by the other guide RNA upon such recognition.

9. A cell of a plant according to claim 1.

10. The plant according to claim 1, wherein the plant is a monocotyledonous plant.

11. The plant according to claim 10, wherein the plant is selected from a wheat plant, a corn plant, a maize plant, a rice plant, a barley plant, an oat plant, or a sorghum plant.

12. The plant according to claim 1, wherein the plant is a dicotyledonous plant.

13. The plant according to claim 12, wherein the plant is selected from a cotton plant, a soybean plant, a beet plant, a potato plant, a tomato plant, or a Brassica plant.

14. A method for obtaining a plant comprising a genetic determinant associated with a trait, wherein the genetic determinant is located on at least one chromosome of the plant, wherein the genetic determinant is positioned between two chromosomal regions I1.1 and I1.2, wherein each chromosomal region I1.1 and I1.2 is located between 1 cM and 5 cM of the genetic determinant, comprising introducing, in a plant comprising the genetic determinant, one or more transgene(s) comprising:
a genetic construct coding for a dead Cas protein,
a genetic construct allowing transcription of at least one guide RNA associated with the dead Cas protein, wherein the gRNA recognizes a DNA sequence located in the chromosomal region I1.1, and
a genetic construct coding for a Spo11 protein, wherein the Spo11 protein is fused such that the Spo11 protein is directed to the DNA sequence of the region I1.1 recognized by the guide RNA upon such recognition.

15. The method of claim 14, wherein the dead Cas protein is under control of a promoter active during meiosis prophase I.

16. The method of claim 14, wherein the gRNA is transcribed during meiosis prophase I.

17. The method of claim 14, wherein the Spo11 protein is fused to the dead Cas protein.

18. The method of claim 14, wherein the dead Cas protein is a dead Cas9 protein.

19. The method of claim 1, wherein the plant is a monocotyledonous plant.

20. The method of claim 19, wherein the plant is selected from a wheat plant, a corn plant, a maize plant, a rice plant, a barley plant, an oat plant, or a sorghum plant.

21. The method of claim 14, wherein the plant is a dicotyledonous plant.

22. The method of claim 21, wherein the plant is selected from a cotton plant, a soybean plant, a beet plant, a potato plant, a tomato plant, or a Brassica plant.
